(19) United States
(12) Patent Application Publication
(10) Pub. No.: US 2023/0400435 A1
(43) Pub. Date: Dec. 14, 2023

(54) MEASURING DEVICE

(71) Applicant: REVORN CO., LTD., Tokyo (JP)

(72) Inventor: Hiroaki MATSUOKA, Tokyo (JP)

(21) Appl. No.: 18/033,759

(22) PCT Filed: Nov. 2, 2021
(86) PCT No.: PCT/JP2021/040460

§ 371 Date: Apr. 25, 2023

(30) Foreign Application Priority Data
Nov. 4, 2020 (JP) 2020-184741

(51) Int. Cl. G01N 29/24; G01N 1/24

(52) U.S. Cl. CPC G01N 29/2443; G01N 1/24; G01N 2291/021

(57) ABSTRACT

A measuring device is provided that can easily measure odors. The measuring device is provided with a board and a housing. An odor sensor is disposed on the board. The housing accommodates the board and has at least three opening portions.

### CROSS REFERENCE TO RELATED APPLICATIONS

This application is a U.S. National Phase Application under 35 U.S.C. 371 of International Application No. PCT/JP2021/040460, filed on Nov. 2, 2021, which claims priority to Japanese Patent Application No. 2020-184741, filed on Nov. 4, 2020. The entire disclosures of the above applications are expressly incorporated by reference herein.

### BACKGROUND

### Technical Field

The present invention relates to a measurement apparatus.

### Related Art

Odor measurement is often performed by bringing air collected in a container or the like into contact with an odor sensor (see WO 2019/117099 A1).

However, measuring odor by the method disclosed in WO 2019/117099 A1 requires a great deal of time and effort.

In view of the above circumstances, the present invention provides a measurement apparatus capable of easily performing odor measurement.

### SUMMARY

According to an aspect of the present invention, a measurement apparatus is provided. The measurement apparatus comprises a substrate and a housing. The substrate is disposed with an odor sensor. The housing accommodates the substrate and includes at least three openings.

According to an aspect of the present invention, it is possible to easily measure odor.

### BRIEF DESCRIPTION OF THE DRAWINGS

FIG. 1 is a diagram showing a front appearance of a measurement apparatus.

FIG. 2 is a diagram showing a rear appearance of the measurement apparatus.

FIG. 3 is a perspective view showing an appearance of the measurement apparatus.

FIG. 4 is a diagram showing a rear surface of a front side of a housing 1.

FIG. 5 is a diagram showing an example of a substrate accommodated in the housing 1.

FIG. 6 is a diagram showing a flow of air/gas according to a first utilization embodiment.

FIG. 7 is a diagram showing a flow of air/gas according to a second utilization embodiment.

### DETAILED DESCRIPTION

Hereinafter, embodiment of the present invention will be described with reference to the drawings. Various features described in the embodiment below can be combined with each other.

A program for realizing a software in the present embodiment may be provided as a non-transitory computer readable medium that can be read by a computer or may be provided for download from an external server or may be provided so that the program can be activated on an external computer to realize functions thereof on a client terminal (so-called cloud computing).

In the present embodiment, the “unit” may include, for instance, a combination of hardware resources implemented by a circuit in a broad sense and information processing of software that can be concretely realized by these hardware resources. Further, various information is performed in the present embodiment, and the information can be represented by, for instance, physical values of signal values representing voltage and current, high and low signal values as a set of binary bits consisting of 0 or 1, or quantum superposition (so-called qubits), and communication/calculation can be performed on a circuit in a broad sense.

Further, the circuit in a broad sense is a circuit realized by combining at least an appropriate number of a circuit, a circuitry, a processor, a memory, or the like. In other words, it is a circuit includes application specific integrated circuit (ASIC), programmable logic device (e.g., simple programmable logic device (SPLD), complex programmable logic device (CPLD), field programmable gate array (FPGA)), or the like.

1. Appearance of the Measurement Apparatus

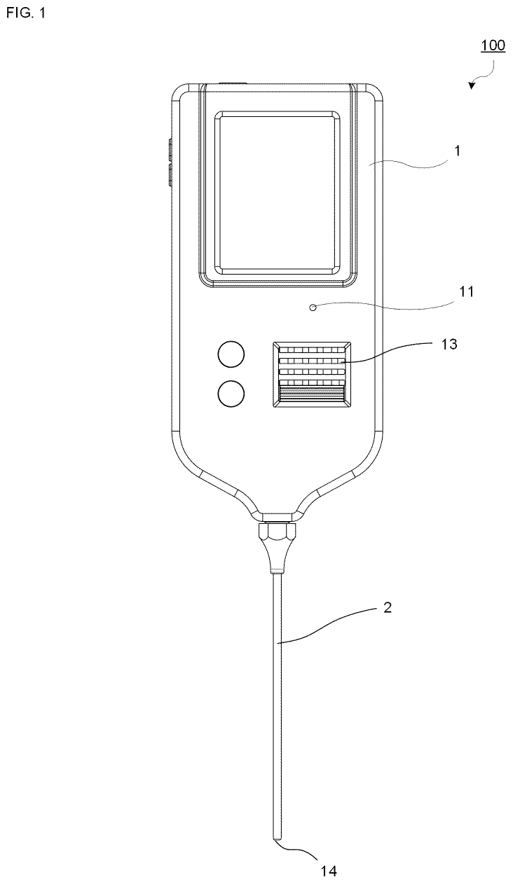

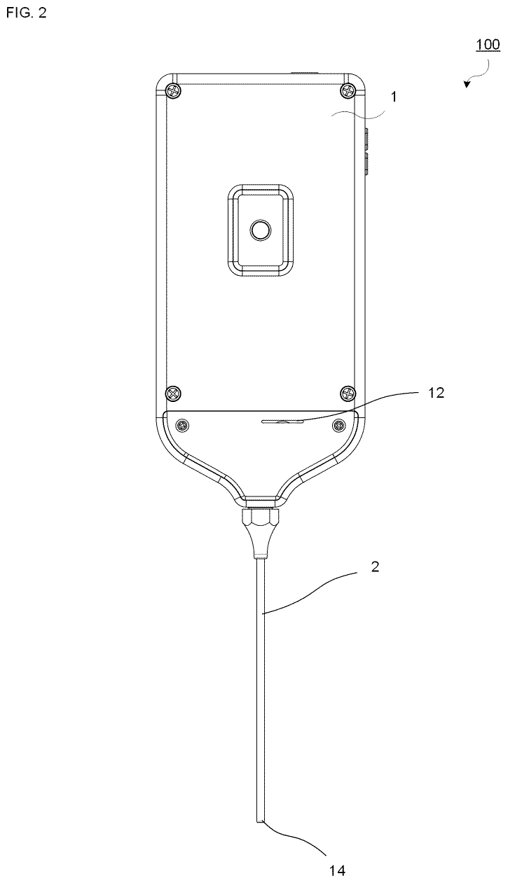

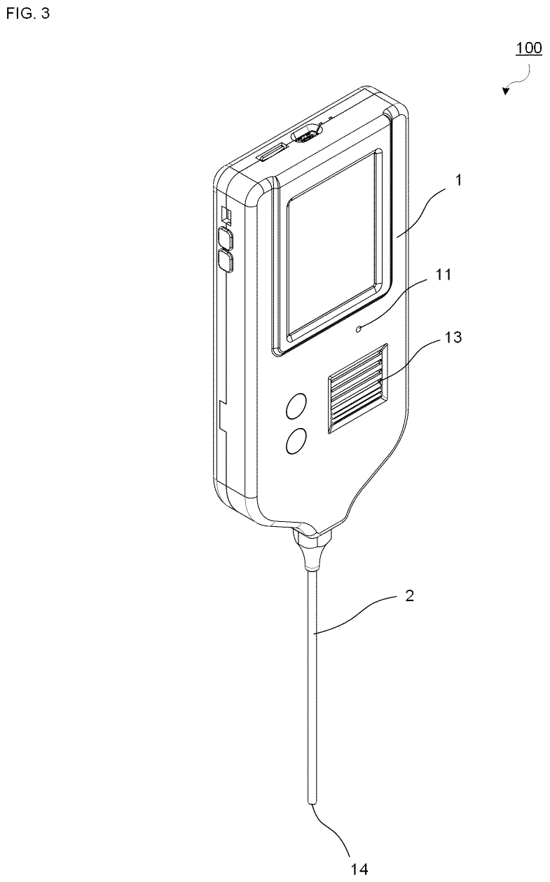

FIG. 1 is a diagram showing a front appearance of a measurement apparatus. FIG. 2 is a diagram showing a rear appearance of the measurement apparatus. Further, FIG. 3 is a perspective view showing an appearance of the measurement apparatus.

A measurement apparatus 100 shown in these drawings comprises a substrate 3 (not shown) and a housing 1. The housing 1 is connected to a nozzle 2. The housing 1 accommodates the substrate (not shown) and include at least three openings. For instance, the housing 1 includes an opening 11 that is a first opening, an opening 12 that is a second opening, an opening 13 that is a third opening, and a nozzle opening 14. Further, two or more third openings may be provided in the housing 1, and at least one of the third openings, e.g., the opening 13, may include a shutter that can be opened/closed.

The opening 11 is an air outlet. The opening 12 is an air inlet. The opening 13 and the nozzle opening 14 are inlets for gas containing a component to be measured.

2. Inside of Housing 1

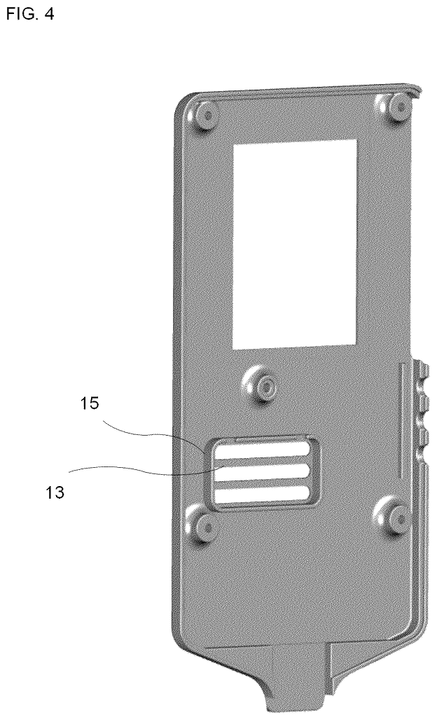

FIG. 4 is a diagram showing a rear surface of a front side of the housing 1. As shown in the drawing, an enclosure 15 may be provided on a rear surface of the opening 13. The enclosure 15 allows gas flowing from the opening 13 to flow in a vicinity of an odor sensor accommodated in the housing 1.

3. Substrate of Measurement Apparatus

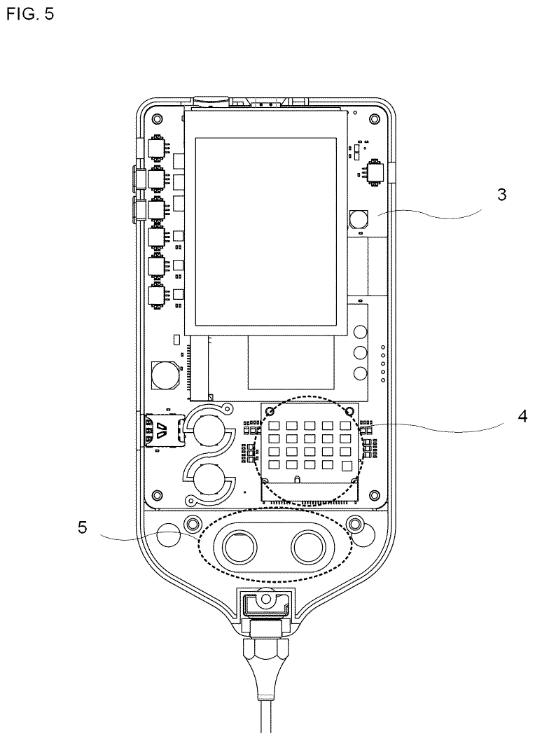

FIG. 5 is a diagram showing an example of a substrate accommodated in the housing 1. As shown in the drawing, the substrate 3 is provided with an odor sensor 4 and a pump 5. The odor sensor 4 is configured of, for instance, two or more Quartz Crystal Microbalance sensors. Further, the odor sensor 4 is arranged directly below the opening 13. The pump 5 allows gas that flows in through the opening 13 and gas that flows in through the nozzle opening 14 to be discharged from the opening 11. The odor sensor 4 may be applied with a semiconductor type such as an oxide semiconductor type or an organic semiconductor type, a quartz oscillator type using an epoxy resin film, a vinyl acetate resin film, a Langmuir-Blodgett film, or the like as a sensitive film, or other types such as using a surface acoustic wave (SAW) filter or a film bulk acoustic wave (FBAR) filter.

4. First Utilization Embodiment of Measurement Apparatus

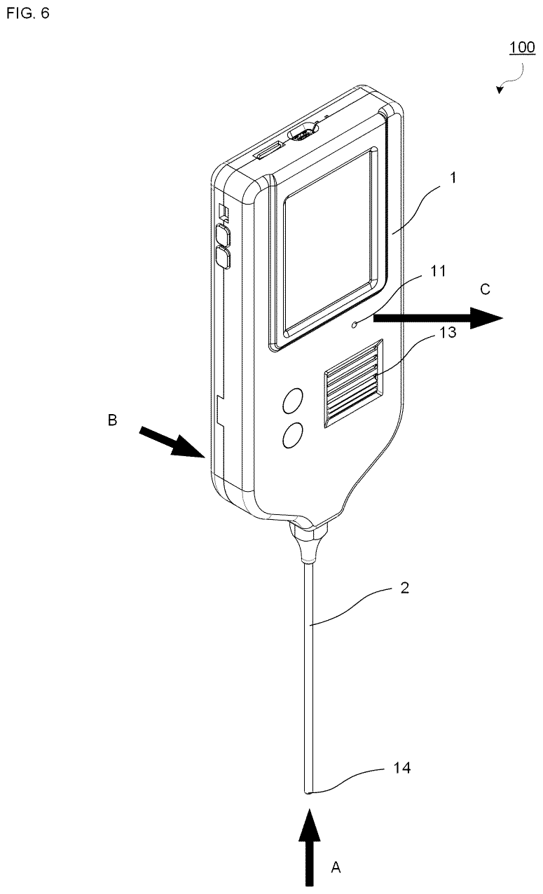

FIG. 6 is a diagram showing a flow of air/gas according to a first utilization embodiment. In the first utilization embodiment, the nozzle opening 14 is an inlet for gas containing a component to be measured, the opening 12 is an inlet for ambient air, and the opening 11 is an outlet for air. Specifically, in the utilization embodiment, the nozzle opening 14 is aimed at an object to be measured by a measurer, and air flows in from the nozzle opening 14 and flows out from the opening 11. To create such a flow of air, the substrate 3 accommodated in the housing 1 may comprise a pump 5. The pump 5 is, for instance, a piezo pump, which allows air that flows in through the opening 12 and the nozzle opening 14 to be discharged from the opening 11. In the utilization embodiment, odor in a vicinity of the measurement apparatus can be measured. At this time, the shutter of the opening 13 is desired to be closed. As a result, in the first utilization embodiment, gas that flows in as indicated by arrow A shown in the drawing and air that flows in as indicated by arrow B shown in the drawing flow out as indicated by arrow C shown in the drawing.

5. Second Utilization Embodiment of Measurement Apparatus

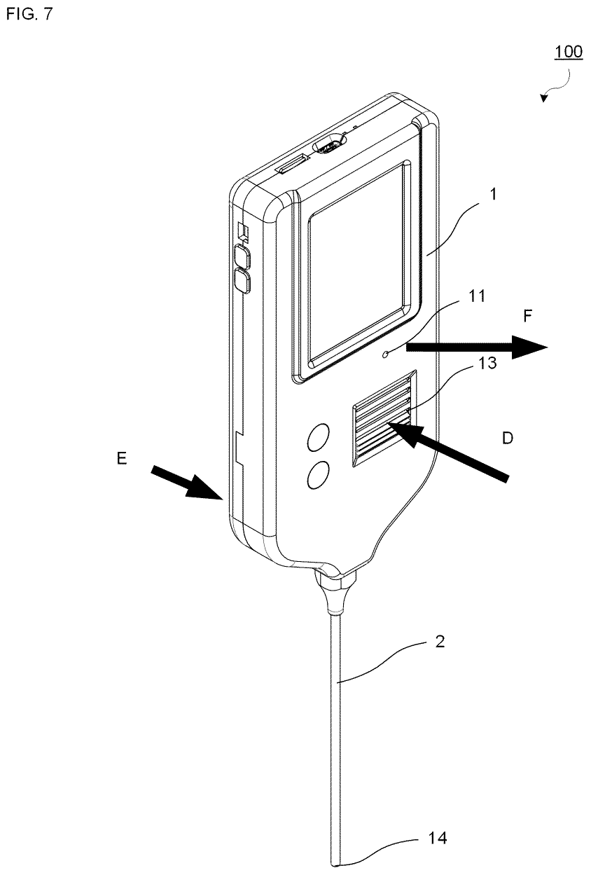

FIG. 7 is a diagram showing a flow of air/gas according to a second utilization embodiment. In the second utilization embodiment, the opening 13 is an inlet for gas containing the component to be measured, the opening 12 is an inlet for ambient air, and the opening 11 is an outlet for air. Specifically, in the utilization embodiment, atmosphere around the measurement apparatus 100 flows in through the opening 13, ambient air (for cleaning the odor sensor 4) flows in through the opening 12, and air flows out through the opening 11. To create such an air flow, the substrate 3 accommodated in the housing 1 may comprise a pump The pump 5 is, for instance, a piezo pump, which allows air that flows in through the opening 12 and the opening 13 to be discharged from the opening 11. In the utilization embodiment, odor in a vicinity of the measurement apparatus 100 can be measured. As a result, in the second utilization embodiment, gas that flows in as indicated by arrow C shown in the drawing and air that flows in as indicated by arrow D shown in the drawing flow out as indicated by arrow E shown in the drawing.

6. Other

The present invention may be provided in each of the following aspects.

The measurement apparatus, wherein: the housing includes a first opening that is an air outlet, a second opening that is an air inlet, and a third opening that is an inlet for gas containing a component to be measured.

The measurement apparatus, wherein: the substrate includes a pump configured to allow air that flows in through the third opening to be discharged from the first opening.

The measurement apparatus, wherein: two or more third openings are provided in the housing.

The measurement apparatus, wherein: at least one of the third openings includes a shutter that can be opened/closed.

The measurement apparatus, wherein: the odor sensor is configured of two or more Quartz Crystal Microbalance sensors.

Of course, the present invention is not limited to the above aspects.

What is claimed is:

1. A measurement apparatus, comprising:
a substrate disposed with an odor sensor; and
a housing accommodating the substrate and including at least three openings.

2. The measurement apparatus according to claim 1, wherein:
the housing includes
a first opening that is an air outlet,
a second opening that is an air inlet, and
a third opening that is an inlet for gas containing a component to be measured.

3. The measurement apparatus according to claim 2, wherein:
the substrate includes a pump configured to allow air that flows in through the third opening to be discharged from the first opening.

4. The measurement apparatus according to claim 2, wherein:
two or more third openings are provided in the housing.

5. The measurement apparatus according to claim 4, wherein:
at least one of the third openings includes a shutter that can be opened/closed.

6. The measurement apparatus according to claim 1, wherein:
the odor sensor is configured of two or more Quartz Crystal Microbalance sensors.
